(19) United States
(12) Patent Application Publication
(10) Pub. No.: US 2026/0142032 A1
(43) Pub. Date: May 21, 2026

(54) SYSTEM AND METHOD FOR FEDERATED LEARNING AMONG MEDICAL INSTITUTIONS, AND DISEASE PROGNOSIS SYSTEM INCLUDING SAME

(71) Applicant: NATIONAL CANCER CENTER, Goyang-si (KR)

(72) Inventors: Hyun Woo PARK, Goyang-si (KR); Kyoung Yeon BACK, Seoul (KR); Jae Dong LEE, Goyang-si (KR); Hyo Soung CHA, Goyang-si (KR); Yu Min KIM, Goyang-si (KR); Ye Ji LEE, Goyang-si (KR)

(73) Assignee: NATIONAL CANCER CENTER, Goyang-si (KR)

(21) Appl. No.: 19/124,762

(22) PCT Filed: Sep. 20, 2023
(86) PCT No.: PCT/KR2023/014218

§ 371 Date: Apr. 25, 2025

(30) Foreign Application Priority Data
Oct. 27, 2022 (KR) 10-2022-0140554

(51) Int. Cl. G16H 50/20

(52) U.S. Cl. CPC G16H 50/20

(57) ABSTRACT

A federated learning system and method among medical institutions, and a disease prognosis prediction system including the same. The federated learning system and method are configured to apply a hierarchical clustering-based learning method during federated learning using medical data, and to transmit weights generated based on machine learning results by applying quantum cryptography and timestamp-based encryption techniques. The system and method enable resolution of data heterogeneity among medical institutions, thereby improving the performance of the learning model, and ensures stability by providing protection of personal medical data.

### TECHNICAL FIELD

The present invention relates to a federated learning system and method that constructs different data formats across medical institutions into a unified data format to overcome data heterogeneity among medical institutions and enhance the protection of personal information in medical data, and to a disease prognosis prediction system incorporating such a federated learning system.

### BACKGROUND ART

Due to the limitations of medical resources in clinical settings, the adoption of artificial intelligence (AI)-based interpretation and diagnostic support systems within medical institutions has gained increasing attention. Achieving high-performance medical services through AI requires training on large volumes of high-quality source data. However, strict data privacy regulations often prevent medical data from being shared externally, restricting model training to the limited datasets available within a single medical institution. This constraint poses significant challenges in delivering enhanced medical services.

Without centralizing data, a learning paradigm known as federated learning has been proposed, which enables the training of deep neural networks (DNNs) using medical data distributed across multiple locations.

Federated learning offers the advantage of training on a more extensive dataset than what a single medical institution alone can provide. However, to achieve this, it is necessary to address performance degradation issues arising from data heterogeneity and non-uniformity among different medical institutions. Specifically, each medical institution may utilize different event codes, making it challenging to integrate events related to the same patient who has visited multiple institutions. Furthermore, it is difficult to immediately utilize events conducted at different medical institutions, and redundant execution of the same event may occur, thereby hindering the effective prediction of diseases.

Additionally, in federated learning, only the weights from the training results is transmitted, which provides a certain level of protection against personal data leakage. However, there remains a concern that personal information may still be inferred from such weights. Specifically, the weights of the training results may be reverse-traced to deduce the raw data, necessitating the implementation of additional security measures to ensure the protection of personal information.

### DETAILED DESCRIPTION OF THE INVENTION

### Technical Problem

The objective of the present invention is to address the aforementioned issues by providing a federated learning system and method capable of mitigating the heterogeneity arising from differences in data structures among medical institutions, thereby maximizing the performance of machine learning models.

Another objective of the present invention is to provide a federated learning system and method that allows for the enhancement of the security of personal information in federated learning.

Another objective of the present invention is to provide a system that allows for more accurate disease prognosis prediction by utilizing a federated learning system that mitigates the heterogeneity of medical data and enhances the protection of personal information.

The technical problems addressed by the present invention are not limited to those mentioned above, and additional technical problems not explicitly stated will be readily understood by those skilled in the art from the following description.

### Means for Solving the Problem

To achieve the above objective, a federated learning system among medical institutions includes: a plurality of local servers provided in medical institutions; and a global server configured to communicate with the local servers. Each of the local servers comprises a weight update unit configured to update its own machine learning model using weights from other local servers. The weight update unit is configured to apply weights differently based on whether medical data collected by each of the medical institutions follows an independent identically distributed distribution or a non-independent identically distributed distribution.

The non-independent identically distributed distribution may include cases in which: same data variables do not follow a uniform distribution across the medical institutions; a difference in distribution between normal group and disease group for a target condition is observed across the medical institutions, such that the data does not follow a uniform distribution; distribution of age (x) given disease (y) or distribution of disease (y) given age (x), based on conditional probability, does not follow a uniform distribution across medical institutions; or an amount of data collected across medical institutions exhibits non-uniform distribution characteristics.

The weight update unit may be configured to update weights using a hierarchical clustering method when the collected medical data exhibits characteristics of a non-independent identically distributed distribution. the hierarchical clustering method may include: calculating similarity between local servers using a first equation,

$$\text{?}$$

$$\text{?} \text{indicates text missing or illegible when filed}$$

to cluster local weights having similar data distributions; calculating similarity between different clusters using a second equation,

$$\text{?}$$

$$\text{?} \text{indicates text missing or illegible when filed}$$

to merge similar clusters; and updating weights within each of the clusters using a third equation,

$$\text{?}$$

$$\text{?} \text{indicates text missing or illegible when filed}$$

The weight update unit may be configured to update the weights of the machine learning model trained at each of the local servers using

$$\text{?}$$

$$\text{?} \text{indicates text missing or illegible when filed}$$

when the collected medical data exhibits characteristics of an independent identically distributed distribution.

The local server may further include a cryptographic key generation unit that utilizes a quantum cryptographic key and a timestamp code. The cryptographic key generation unit may be configured to generate a time-based secret key and a time-based public key by respectively combining the timestamp code with a private key and a public key. The time-based public key may be transmitted to the global server.

A quantum key generation and distribution device may be connected to at least one of the local server and the global server via a quantum key management device. The quantum key generation and distribution device may be configured to provide the quantum cryptographic key to the quantum key management device.

The local server further may include a personal information protection unit configured to group weights generated based on a machine learning result and a hash value, encrypt the grouped data using the quantum cryptographic key, and encrypt the quantum cryptographic key using the time-based secret key.

The timestamp code may include: a weight occurrence time; and communication time information for performing communication with the global server.

The global server may be configured to: compare the timestamp code with an actual reception time of the time-based public key to authenticate the time-based public key; decrypt the quantum cryptographic key using the authenticated time-based public key; and decrypt the weights and the hash value using the decrypted quantum cryptographic key to obtain the weights and the hash value.

The local server may further include: a data acquisition unit configured to acquire medical data; a common data model construction unit configured to transform heterogeneous data structures specific to each medical institution into a standardized model; a data preprocessing unit configured to preprocess data required for machine learning from among data constructed based on the common data model; and a learning unit configured to perform machine learning on the preprocessed data using the machine learning model.

In another general aspect of the present invention, a disease prognosis prediction system using the federated learning system includes: the federated learning system according to any one of claims 1 to 10; and a terminal device configured to interact with the local server. The disease prognosis prediction system is configured to analyze and predict a prognosis of a patient's disease based on the machine learning result.

The terminal device may include: a patient query information input unit; an EMR interfacing and retrieval unit configured to interwork with an EMR backup server within a medical institution to retrieve patient's historical health information; a PHR interfacing unit including: a cancer screening questionnaire interfacing unit, an Internet of Medical Things (IoMT) device interfacing unit configured to acquire health status information using a IoMT device, and a self-input unit for manually inputting health information; a first display unit configured to analyze, process, and output disease risk level information predicted based on personalized health screening data; and a second display unit configured to provide personalized medical content information based on the patient's customized health information.

In another general aspect of the present invention, a federated learning method among medical institutions, in which a federated learning system comprising local servers and a global server transmits and receives medical data for federated learning, the method comprising: distributing, by a quantum key generation and distribution device, a quantum cryptographic key to a local server and the global server via a quantum key management device; generating, by the local server, a time-based secret key and a time-based public key by respectively combining a timestamp code with a private key and a public key; performing, by the local server, machine learning on the medical data using a machine learning model, and generating weights; grouping, by the local server, original weights of the generated weights and a hash value, and encrypting the grouped data using the quantum cryptographic key; encrypting, by the local server, the quantum cryptographic key using the time-based secret key; and transmitting, by the local server, the encrypted original weights and the hash value to the global server.

The federated learning method among medical institutions may include, by the global server, authenticating a time-based public key transmitted by the local server; decrypting, when the time-based public key is successfully authenticated, the quantum cryptographic key using the time-based public key; decrypting the original weights and the hash value using the decrypted quantum cryptographic key to obtain the original weights and the hash value; calculating a hash value of the original weights using the time-based public key, performing a comparison operation between the calculated hash value and a hash value received from the medical institution to authenticate the original weights, and updating the weights after the authentication; and transmitting the weights to the local server to allow the local server to update the machine learning model.

The authenticating the time-based public key by the global server may be performed by comparing the timestamp code with an actual reception time of the time-based public key.

The federated learning method among medical institutions may further include: applying, by the local server, different weights based on whether the medical data collected by each medical institution follows an independent identically distributed distribution or a non-independent identically distributed distribution.

The local server may update the weights using a hierarchical clustering method when the collected medical data exhibits characteristics of a non-independent identically distributed distribution. The hierarchical clustering method may include: calculating similarity between local servers using a first equation,

$$\text{?}$$

$$\text{?} \text{indicates text missing or illegible when filed}$$

to cluster local weights with similar data distributions; calculating similarity between different clusters using a second equation,

$$\frac{\text{?}}{\text{?}} - \text{?}$$

$$\text{?} \text{indicates text missing or illegible when filed}$$

to merge similar clusters; and updating weights within each cluster using a third equation,

$$\text{?} {\sum }_{\text{?}}^{\text{?}} \frac{\text{?}}{\text{?}} \text{?}$$

$$\text{?} \text{indicates text missing or illegible when filed}$$

When the collected medical data exhibits characteristics of an independent identically distributed distribution, the local server may update the weights of the machine learning models trained by each local server using

$$\text{?} {\sum }_{\text{?}}^{\text{?}} \frac{\text{?}}{\text{?}} \text{?}$$

$$\text{?} \text{indicates text missing or illegible when filed}$$

### Effects of the Invention

According to the present invention, by applying a hierarchical clustering learning method in federated learning using medical data, it is possible to effectively address issues that may arise in non-independent identically distributed scenarios, which vary across different medical institutions.

According to the present invention, it is possible to compensate for the structural and terminological heterogeneity of medical data across different medical institutions, thereby enhancing the reliability of federated learning results.

According to the present invention, by applying quantum cryptography and timestamp code encryption methods to the weights transmitted to the global server based on machine learning results during federated learning, it is possible to completely eliminate the possibility of reconstructing the weights to infer the raw data. Accordingly, the invention enhances the stability of data protection in a federated learning environment and provides verified and reliable federated learning results.

### BRIEF DESCRIPTION OF DRAWINGS

FIG. 1 is a diagram illustrating the overall system configuration for federated learning among medical institutions according to a preferred embodiment of the present invention.

FIG. 2 is a detailed configuration diagram of a local server provided in a medical institution.

FIG. 3 is an overall flowchart illustrating a federated learning method according to the present invention.

FIG. 4 is a flowchart illustrating the process of encrypting weights transmitted between the local server and the global server during the federated learning process according to the present invention.

FIG. 5 is a flowchart providing a detailed explanation of the federated learning process described in FIG. 4. Specifically, FIG. 5A is a flowchart illustrating the federated learning process between Medical Institution A and the global server, while FIG. 5B is a flowchart illustrating the federated learning process between Medical Institution B and the global server.

FIG. 6 is a configuration diagram of a terminal device that interacts with local servers (100a to 100n) according to an embodiment of the present invention.

FIG. 7 is a configuration diagram of a user interface screen displayed on the first display unit (240) according to the present invention.

FIG. 8 is a configuration diagram of a user interface screen displayed on a second display unit (250) according to the present invention.

### BEST MODE FOR CARRYING OUT THE INVENTION

The present invention is capable of various modifications and may have multiple embodiments. Specific embodiments are illustrated in the drawings and described in detail herein. However, these are not intended to limit the invention to particular embodiments, but rather should be understood to encompass all modifications, equivalents, and substitutes that fall within the spirit and scope of the invention. In describing the present invention, detailed explanations of well-known technologies may be omitted when it is determined that such descriptions could obscure the essence of the invention.

The terms “first,” “second,” and the like may be used to describe various components; however, these components should not be limited by such terms. These terms are used solely for the purpose of distinguishing one component from another.

The terminology used in the present invention is intended solely for the purpose of describing specific embodiments and is not intended to limit the invention. Unless explicitly stated otherwise in context, singular expressions include plural forms as well. In this application, terms such as “include” or “have” are intended to specify the presence of the stated features, numbers, steps, operations, components, parts, or combinations thereof, but should not be construed as excluding the possibility of the presence or addition of one or more other features, numbers, steps, operations, components, parts, or combinations thereof.

Spatially relative terms such as “below,” “beneath,” “lower,” “above,” and “upper” may be used to describe the relationship between one element or component and another, as illustrated in the drawings, to facilitate explanation. These spatially relative terms should be understood to include different orientations of the elements during use or operation, in addition to the directions shown in the drawings. For example, if a component shown in the drawings is inverted, an element described as being “below” or “beneath” another element may, in fact, be positioned “above” that element. Accordingly, the exemplary term “below” may encompass both upward and downward directions. Components may be oriented in different directions, and thus, spatially relative terms should be interpreted accordingly based on their orientation.

The terms such as “unit” or “portion” used in the present invention, which indicate a part of a component, may refer to a device capable of performing a specific function, software capable of performing a specific function, or a combination of a device and software capable of performing a specific function. However, these terms are not necessarily limited to the explicitly stated functions. They are provided merely to facilitate a broader understanding of the invention. A person skilled in the art to which the present invention pertains would understand that various modifications and variations can be made based on these descriptions.

Additionally, all electrical signals used in the present invention are provided as examples. It should be noted that if an inverter or similar component is additionally included in the circuit of the present invention, the polarity of all electrical signals described herein may be reversed. Accordingly, the scope of the present invention is not limited to the direction of the signals.

Accordingly, the spirit of the present invention should not be construed as being limited to the described embodiments. Rather, all modifications and variations that are equivalent or equivalent substitutions within the scope of the following claims shall be considered to fall within the scope of the spirit of the present invention.

Hereinafter, the present invention will be described in further detail based on the embodiments illustrated in the drawings.

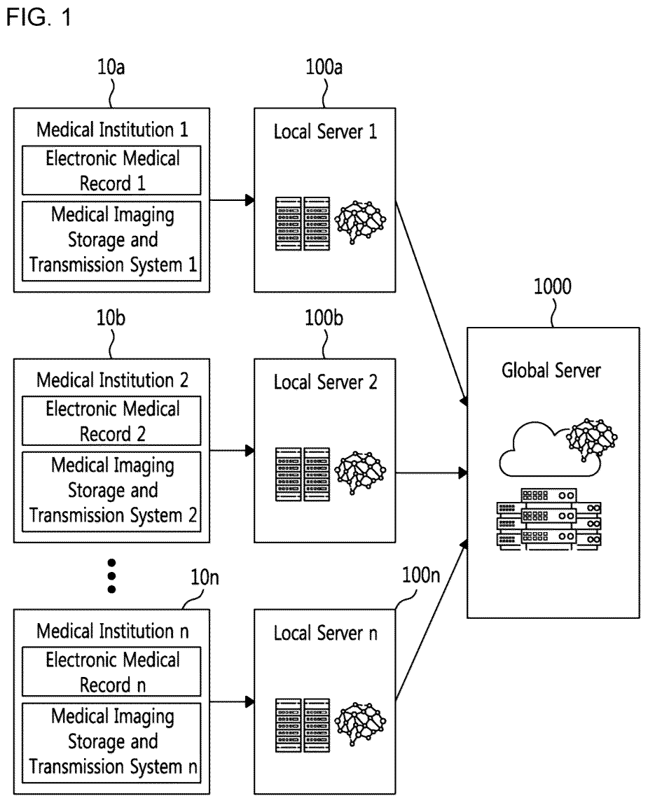

FIG. 1 is a diagram illustrating the overall system configuration for federated learning of medical data according to a preferred embodiment of the present invention.

Referring to FIG. 1, the system includes the first to n-th medical institutions (10a to 10n), local servers (100a to 100n) provided in each medical institution (10a to 10n), and a global server (1000, also referred to as a central server) that communicates with the local servers (100a to 100n).

Each medical institution (10a to 10n) includes an electronic medical record (EMR) system that records and processes all health information related to patient visits, including diagnosis, treatment, and surgery. The EMR system stores and processes all medical data related to a patient's clinical care in a database and is also capable of generating new information. To support the EMR system, each medical institution is typically equipped with electronic devices for processing medical data, as well as backup servers.

The medical data processed by the medical institutions (10a to 10n) can be classified into electronic medical record (EMR) data and medical imaging data, which will be described in detail below.

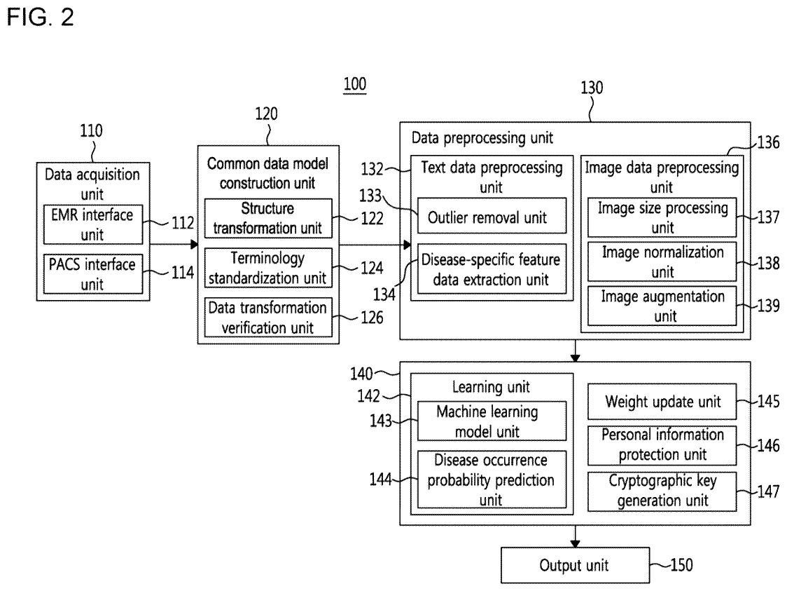

The local servers (100a to 100n) are installed within the medical institutions (10a to 10n) and perform machine learning based on patient medical information to analyze and predict the prognosis of a patient's disease, providing the prediction results. The configuration of the local servers (100a to 100n) is described in detail in FIG. 2.

The global server (1000) communicates with the local servers (100a to 100n) to receive machine learning models and weights and functions to update the machine learning models of the local servers (100a to 100n). In this embodiment, the machine learning model refers to an artificial intelligence model that is trained using a large set of medical data and health information data through a series of learning algorithms to achieve specific objectives, such as predicting disease onset probability or mortality rates.

FIG. 2 is a detailed configuration diagram of a local server installed in a medical institution.

Referring to FIG. 2, the local server (100a to 100n) may be configured to include a data acquisition unit (110), a common data model construction unit (120), a data preprocessing unit (130), a learning unit (142), a weight update unit (145), a personal information protection unit (146), a cryptographic key generation unit (147), and an output unit (150).

In FIG. 2, the data acquisition unit (110) is a unit that acquires various medical data, including treatment, prescriptions, and other related information, from medical institutions (10a to 10n). The medical data acquired by the data acquisition unit (110) may include electronic medical record (EMR) data and medical imaging data. Both data types or each one separately can be obtained in a de-identified manner.

The medical data managed by the medical institutions (10a to 10n) can be classified into text-based EMR data and medical imaging data. Accordingly, the data acquisition unit (110) includes an EMR interfacing unit (112) for extracting and storing text-based data and a PACS interface unit (114) for extracting and storing imaging data.

EMR data may include, for example, a dataset containing any of the following: cancer registration information for cancer patients, anticancer drug treatment records, radiation therapy records, surgical treatment records, or diagnostic test results. The EMR data can be acquired in any of the following formats: Relational Database (RDB), Excel, JSON, or XML.

Medical imaging data may include an image dataset containing images from either Computer Tomography (CT) or Magnetic Resonance Imaging (MRI). The image data can be acquired in any of the following formats: DCM (DICOM) or PNG. Additionally, utilizing such medical imaging data enables the provision of diagnostic assistance information, such as body surface area, muscle mass, and abdominal muscle mass.

Since each medical institution (10a to 10n) may have different data structures and medical terminology codes depending on the type of medical system used, it is necessary to establish a standardized model that can be applied universally across all medical institutions. The common data model construction unit (120) is essential for addressing the data heterogeneity issues among medical institutions (10a to 10n) and preventing biased federated learning results.

In FIG. 2, the common data model construction unit (120) includes a structure transformation unit (122), a terminology standardization unit (124), and a data transformation verification unit (126).

The structure transformation unit (122) transforms the data received from the data acquisition unit (110) into a common data structure model by referencing the common model schema, enabling interoperability. Additionally, it allows for the addition and modification of data structures to accommodate items not included in the common data structure model, ensuring expandability.

The structure transformation unit (122) may have a data transformation structure that allows for expansion based on both common items shared across different cancer types and characteristics specific to each cancer type. The data transformation structure may include common items such as patient basic information (including gender, birth year/month, and cancer history); patient health information (including alcohol consumption, smoking history, and family medical history); patient anthropometric data (including height, weight, and body mass index (BMI)); diagnosis information (including diagnosis date and diagnosis code); diagnostic test information (including test date, test code, and test results); imaging test information (including imaging test date, imaging test code, and imaging test findings); surgical information (including surgery date, surgery EDI code, surgery duration, and intraoperative blood loss); and medication information (including prescription date, prescription code, dosage, and duration of administration). The structure transformation unit (122) may perform transformation including date, medical terminology codes, prescription codes specific to each medical institution, and result values by referencing the common model schema.

The terminology standardization unit (124) is a unit that maps different medical terminology codes used by respective medical institutions (10a to 10n) to international standard clinical terminology through an international standard clinical terminology database. By utilizing the terminology standardization unit (124), the different medical terminology codes used by each medical institution (10a to 10n) are standardized and transformed, thereby enabling transformation into a common data structure and code that can be utilized for learning across multiple medical institutions.

The data transformation verification unit (126) is a unit that verifies the quality of the data constructed into a common data model by the structure transformation unit (122) and the terminology standardization unit (124). Quality indicators for data transformation verification may include completeness, consistency, timeliness, and validity of the data. Based on the results of the data transformation verification, the operation of the common data model construction unit (120) may be repeated.

In FIG. 2, the data preprocessing unit (130) is a unit for preprocessing the data necessary for machine learning from among the data constructed based on the common data model.

The data preprocessing unit (130) may be classified into a text data preprocessing unit (132) and an image data preprocessing unit (136), depending on the type of data to be preprocessed.

The text data preprocessing unit (132) includes an outlier removal unit (133) configured to remove data that is determined to be improperly loaded with the text-related data constructed in the common data model or identified as an outlier based on data distribution, and a disease-specific feature data extraction unit (134) configured to extract significant data required for training an artificial intelligence model based on the disease to be predicted.

The image data preprocessing unit (136) includes: an image size processing unit (137) configured to reduce unnecessary portions of an image by cropping or to increase image size by adding padding to small images to convert them to a uniform size; an image normalization unit (138) configured to perform normalization to remove variations between image data in medical imaging having RGB values, such as pathological images, or to apply a Gaussian filter to enhance image clarity; and an image augmentation unit configured to augment medical image data by applying various filters and performing transformations such as image enhancement and horizontal flipping, in order to prevent overfitting to specific images and improve the performance of the machine learning model. The reason for preprocessing the image data is that resizing is required to adjust the image size so that the video images collected from the data acquisition unit 110 have a consistent size.

In FIG. 2, the learning unit (142) includes: a machine learning model unit (143) configured to provide an optimal prognosis prediction model (i.e., a machine learning model or artificial intelligence model) generated through weight updates; a disease occurrence probability prediction unit (144) configured to perform machine learning to predict the probability of disease onset in a patient, and so on. The optimal prognosis prediction model may be generated by continuously updating weights through iterative communication between the local servers (100a to 100n) and the global server (1000), without sharing medical data from the local servers (100a to 100n).

In FIG. 2, the weight update unit (145) functions to update its own machine learning model by utilizing weights from other local servers. Specifically, the global server (1000) generates a global weight by aggregating local weights from other local servers (i.e., medical institutions). The respective weight update unit of each medical institution (10a to 10n) updates and optimizes its own machine learning model by utilizing the generated global weight.

The weight update unit (145) may update in different ways depending on the distribution characteristics of the medical data collected at each medical institution (10a to 10n). Specifically, the update method varies based on whether the data exhibits independent identically distributed (IID) characteristics or non-independent identically distributed (non-IID) characteristics.

The medical data collected at each medical institution (10a to 10n) does not share the same probability distribution across all institutions (10a to 10n) due to variations in the number of patients and the type of medical equipment used, which affects medical imaging information (e.g., resolution and size). As a result, the data distribution of each medical institution (10a to 10n) exhibits non-independent identically distributed (non-IID) characteristics. Consequently, the prognosis prediction results for each medical institution (10a to 10n) may yield localized outcomes specific to a particular institution rather than generalized results applicable to all medical institutions. Therefore, it is necessary to verify whether the medical data distribution collected from each medical institution (10a to 10n) follows non-IID characteristics. This verification can be conducted based on the following four characteristics.

It is assumed that the data distribution collected based on the data variable x and classified according to the class label y at the i-th medical institution is represented as pᵢ(x,y). For example, if the distribution of acute kidney injury based on the age variable at Medical Institution A is represented as P_{Institution A}(age, acute kidney injury), then the verification of the aforementioned data distribution can be conducted using pᵢ(x) pᵢ(x|y), pⱼ(y|x), and the quantity of data collected at each medical institution.

First, pᵢ(x) is defined as non-independent and identically distributed (non-IID) if missing values or noise occur in the same data variable x collected by each medical institution, resulting in the data variable at each medical institution not following a uniform distribution

Second, pᵢ(y) is defined as non-independent identically distributed (non-IID) if the difference in distribution between normal and diseased subject groups for the target disease to be predicted varies across medical institutions, resulting in the distributions not following a uniform pattern.

Third, pᵢ(x|y), pᵢ(y|x) is defined as non-independent identically distributed (non-ID) if the distribution of age (x) for each disease (y) or the distribution of disease (y) for each age (x) does not follow a uniform distribution across medical institutions, based on conditional probability.

Fourth, it is determined whether the characteristics of the data quantity collected at each medical institution exhibit a non-uniform distribution.

As described above, the present embodiment enables the verification of whether the medical data exhibits non-independent identically distributed (non-IID) characteristics. Based on the verification results, the global weight is updated through a process different from that of independent identically distributed (IID) data. This is necessary to account for the sensitivity of personal information and the accuracy of disease prediction.

Specifically, when the medical data follows a non-independent identically distributed (non-IID) distribution, a hierarchical clustering learning method to mitigate data heterogeneity is applied to update the weights accordingly.

Hierarchical clustering learning clusters local weights

$$( \text{?} , \text{?} )$$

$$\text{?} \text{indicates text missing or illegible when filed}$$

of local servers (100a to 100n) that have similar data distributions. Within the same cluster, the local weights of the local servers (100a to 100n) are first aggregated to update the weights. In this process, the similarity between local servers (100a to 100n) is determined using the equation (1) described below.

$$cos = \frac{w \text{?} \cdot w \text{?}}{‖ w \text{?} ‖ ‖ w \text{?} ‖} \qquad [ Equation 1 ]$$

$$\text{?} \text{indicates text missing or illegible when filed}$$

The similarity between different clusters (A, B) is compared using Equation (2) below. Based on the comparison results, clusters with high similarity are merged into a single cluster through agglomerative clustering. Subsequently, hierarchical clustering is continuously performed, and finally, the weights within the cluster are updated using Equation (3) described below.

$$\frac{{n}_{A} {n}_{B}}{{n}_{A} + {n}_{B}} {‖ {w}_{A} - {w}_{B} ‖}^{2} \qquad [ Equation 2 ]$$

$$w \text{?} ← {\sum }_{k = 1}^{K} \frac{{n}_{k}}{n} w \text{?} \qquad [ Equation 3 ]$$

$$\text{?} \text{indicates text missing or illegible when filed}$$

The hierarchical clustering learning relationship described above can be summarized as follows.

That is, in the hierarchical clustering learning method, the similarity between local servers (100a to 100n) within the same cluster, where the data distributions are similar, is determined using Equation (1) above. The similarity between different clusters (A, B) is determined using Equation (2) above. Finally, the weights within the cluster are updated using Equation (3) above.

Meanwhile, if the medical data collected by each medical institution (100a to 100n) follows an independent identically distributed (IID) distribution, the global server (1000) updates the learned model weights (**) trained at the local servers (100a to 100n) for each round (t). In this case, the weight updates between the local servers (100a to 100n) and the global server (1000) are performed using Equation (3) described above.

In FIG. 2, the personal information protection unit (146) prevents the weight values of the learning results from being externally leaked while communication is performed with the global server (1000). Additionally, it performs a function of verifying the learning result data.

In FIG. 2, the cryptographic key generation unit (147) is a unit configured to enhance the protection of personal information by preventing the possibility of reverse inference of personal information from weights, as previously described. Quantum encryption and timestamp codes are utilized for this purpose.

Specifically, the cryptographic key generation unit (147) generates encrypted time public keys and time private keys to protect personal information. The time private key and time public key are generated by combining a timestamp code with a private key and a public key, respectively. The timestamp code may serve as a communication time code by adding a certain time to the weight occurrence time. That is, the weight occurrence time is measured in nanoseconds (ns, 1/10 billion) at the precision of seconds to create a unique time-based code, and an additional predetermined time is added to the generated time code. Based on the communication time code, the communication time between the local servers (100a to 100n) and the global server (1000) can be calculated.

The process of generating the time private key and time public key can be expressed as follows.

For example, a predetermined time is added to each weight

$$\begin{matrix}w \text{?} , \\ w \text{?} , \\ w \text{?}\end{matrix}$$

$$\text{?} \text{indicates text missing or illegible when filed}$$

to generate a timestamp code as a communication time code, such as 20210603104716.54536708, 20210603104717.10131400, 20210603104717.70181961. The time private key and time public key are then generated by combining this with a private key/public key (RSA encryption) and a communication reservation time code (random number).

In FIG. 2, the learning unit (142), weight update unit (145), personal information protection unit (146), and cryptographic key generation unit (147) may be modularized as a single entity and configured as the control unit (140).

### MODES FOR CARRYING OUT THE INVENTION

Next, the utilization and application of the federated learning system configured as described above will be described.

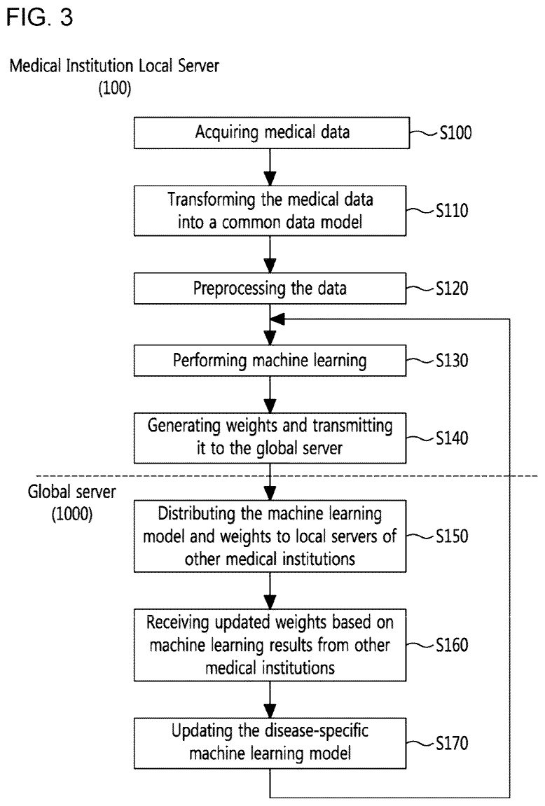

FIG. 3 is an overall flowchart illustrating a federated learning method according to the present invention.

As illustrated in FIG. 3, the process includes the transformation of medical data into a common data model, a preprocessing stage, and a machine learning stage, ultimately providing an optimized disease-specific prognosis prediction model.

According to FIG. 3, the local servers (100a to 100n) provided in the medical institutions (10a to 10n) acquire electronic medical record data and medical imaging data from the medical institutions (10a to 10n) using the data acquisition unit (110) (s100).

Accordingly, the common data model construction unit (120) of the local servers (100a to 100n) constructs a standardized model for electronic medical record data and medical imaging data by utilizing the structure transformation unit (122) and the terminology standardization unit (124) (s110). This process is performed to resolve the heterogeneity issues arising from differences in data structures among medical institutions (10a to 10n). At this time, various common data model (CDM) formats that are internationally applicable, such as OMOP-CDM, Sentinel-CDM, and PCORnet CDM, may be applied to transform the data into a model with a uniform structure and specification. The term “common data model format,” as used herein, refers not only to the standardization of medical terminology but also to a set of predefined rules for constructing a database with an identical structure (e.g., identical schema, table names, column names, etc.). This format may be implemented in the form of an electronic document, such as an Extract, Transform, Load (ETL) specification, or as a program capable of automatically mapping data to a common format. However, the common data model format is not limited to the aforementioned examples and may encompass any format developed independently by a medical institution in a deployable form.

Among the data constructed based on the common data model, preprocessing is performed on the data required for machine learning (s120). The preprocessing process may include the text data preprocessing unit (132) extracting only the data necessary for machine learning, or the image data preprocessing unit (136) transforming imaging data into a form suitable for machine learning. Furthermore, the preprocessing process may include a step in which the disease-specific feature extraction unit (134) extracts meaningful variables for each disease that occur in cancer patients. This is because the data collected by the data acquisition unit (110) may include variables unrelated to the disease or may contain a large number of missing values.

Once the data for machine learning has been preprocessed and provided, the learning unit (142) develops disease-specific machine learning models and performs machine learning based on the preprocessed data (s130). When weight values are generated as a result of machine learning, one of the local servers (local server (100a), for example) transmits the resulting weights to the global server (1000) (s140).

The global server (1000) distributes the disease-specific machine learning model developed by the local server (e.g., 100a), along with the trained model weights, to other local servers (i.e., medical institutions) (100b to 100n) (s150). The other local servers (100b to 100n), upon receiving the disease-specific machine learning model, perform machine learning on their respective locally collected medical data to generate corresponding weights (i.e., local weights), and transmit these local weights to the global server (1000).

The global server (1000) receives the local weights transmitted by the other local servers (100b to 100n), updates the weights according to whether each local server follows an and identically distributed (IID) setting or a non-independent identically distributed (non-IID) setting, and then transmits the updated weights back to the local server (100a) (s160). Accordingly, the local server (100a) may update the disease-specific machine learning model it originally developed based on the weights provided by the other local servers (100b to 100n) (s170).

As such, in this embodiment, the machine learning model is continuously updated using the weights derived from the training results of the other local servers. Consequently, the system can provide an optimized machine learning model whose performance progressively improves beyond that of the initially developed model. Furthermore, the improved machine learning model may also be utilized by the other local servers.

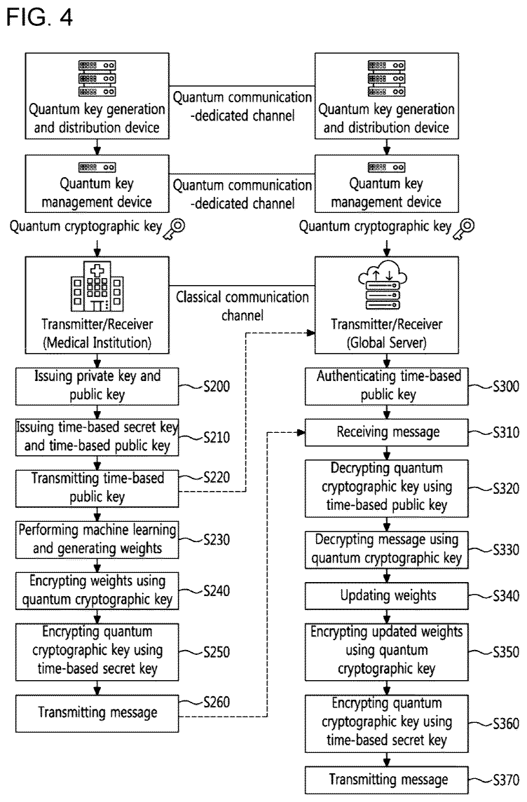

FIG. 4 is a flowchart illustrating a process of encrypting weights transmitted between a local server and a global server during a federated learning process, in accordance with the present invention.

The local server (e.g., medical institution 100a) and the global server (1000) are connected via two communication channels: a quantum communication-dedicated channel and a classical communication channel.

A quantum key generation and distribution device distributes an identical quantum cryptographic key to both the local server (100a) and the global server (1000) via a quantum key management device. At this time, the quantum key generation and distribution device may be provided in at least one of the local server (100a) or the global server (1000). In such a case, any one of the quantum key generation and distribution device may provide the quantum cryptographic key-originally supplied to the local server (100a) or the global server (1000) via the quantum key management device-to the global server (1000) or the local server (100a), respectively, through the quantum communication-dedicated channel.

The cryptographic key generation unit (147) of the local server (100a) is configured to generate cryptographic keys. Specifically, the cryptographic key generation unit (147) issues a private key and a public key (s200), and generates a time-based private key and a time-based public key by combining a communication time code with the private key and the public key, respectively (s210). As previously described, the communication time code includes information indicating the time at which the weight was generated. The time-based public key may be transmitted in advance to the global server (1000) to enable the execution of an authentication procedure (s220).

The learning unit (142) of the local server (100a) performs machine learning based on the preprocessed data, as described in FIG. 3, and generates weights corresponding to the learning results (s230).

Subsequently, the personal information protection unit (146) groups the original weights together with a hash value of the weights, and then encrypts the grouped data using the quantum cryptographic key (s240). Quantum encryption is considered the most secure encryption method from a data security standpoint, as it detects the presence of a malicious third party by inducing a change in the quantum state upon unauthorized intervention, and immediately alters the information accordingly. However, conventional encryption systems are required to provide integrity, confidentiality, authentication, and non-repudiation. Quantum cryptographic keys, by themselves, offer only confidentiality and face limitations in addressing institutional authentication and non-repudiation. In federated learning, institutional authentication is required when transmitting weights to ensure that communication occurs only with authorized institutions.

Accordingly, in the present embodiment, a quantum cryptographic key is encrypted using a time-based secret key generated by a local server (100a), for the purposes of institutional authentication and non-repudiation (s250).

The local server (100a) transmits to the global server (1000) a message encrypted with the time-based secret key, namely, the original weights encrypted with the quantum cryptographic key and the hash value (s260).

The global server (1000), which communicates with the local server (100a), authenticates the time-based public key previously transmitted by the local server (100a) (s300). The authentication of the time-based public key may be a process of verifying whether the time-based public key was indeed transmitted by the local server (100a), by comparing the communication time code of the time-based public key with the actual time of communication. If the result of such authentication indicates a discrepancy, the time-based public key is recognized as an attack key sent by a third party and is invalidated. In another example of invalidation, the time-based public key may also be rendered invalid if an error in calculating the communication time occurs during the process of generating the time-based public key at the local server (100a), resulting in transmission either earlier or later than the scheduled time. When the time-based public key is invalidated in this manner, the global server (1000) requests a new time-based public key from the local server (100a), and the local server (100a) is required to recalculate the time code for communication, regenerate the time-based public key, and transmit it to the global server (1000).

When the global server (1000) receives the original weights and the hash value encrypted with the quantum cryptographic key (s310), it decrypts the quantum cryptographic key-previously encrypted by the local server (100a)—using the authenticated time-based public key (s320). Then, using the decrypted quantum cryptographic key, the global server (1000) decrypts the message, namely, the original weights and the hash value encrypted with the quantum cryptographic key (s330). Through this decryption process, the global server (1000) is able to obtain the original weights and the hash value. Thereafter, the global server (1000) calculates a hash value of the original weights using the time-based public key, performs a comparison operation with the hash value provided by the medical institution to authenticate the raw data, and then updates the weights accordingly (s340).

When the weights is updated, the global server (1000) transmits the updated weights to the local server (100a), thereby optimizing the machine learning model developed by the medical institution. At this time, in order to ensure information security, the updated weights should be transmitted in an encrypted state. Accordingly, the global server (1000) encrypts the updated weights using the previously provided quantum cryptographic key (s350), and subsequently encrypts the quantum cryptographic key using a time-based secret key (s360), before transmitting the result to the local server (100a) (s370).

Then, as described above, the local server (100a) decrypts the quantum cryptographic key using the time-based public key, and subsequently decrypts the encrypted, updated weights using the quantum cryptographic key. Once the updated weights have been successfully decrypted, they are applied to the machine learning model.

As such, the present invention enables a plurality of local servers (medical institutions) (100a to 100n) and a global server (1000) to continuously communicate and perform federated learning, wherein the weights is transmitted and received in an encrypted form using quantum cryptography and time-stamping. This configuration completely eliminates the possibility of inferring personal information by backtracking the weights, as was possible in conventional systems.

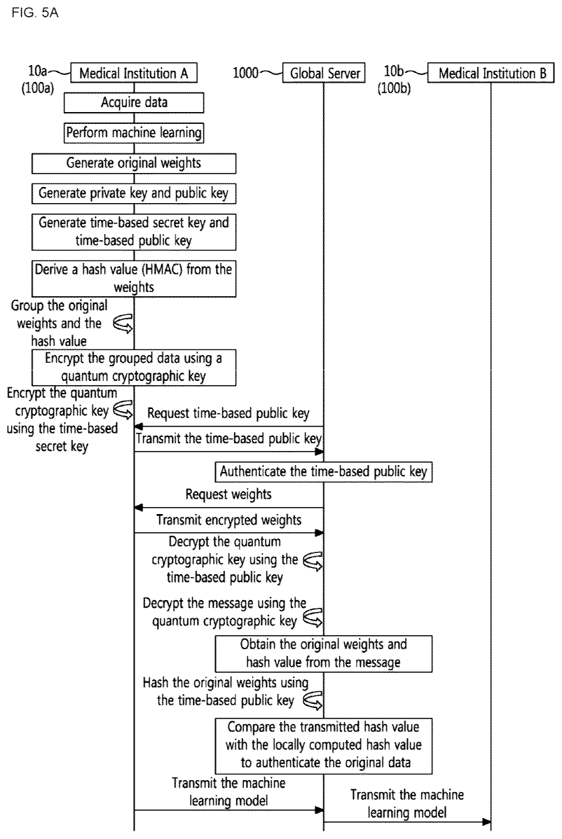

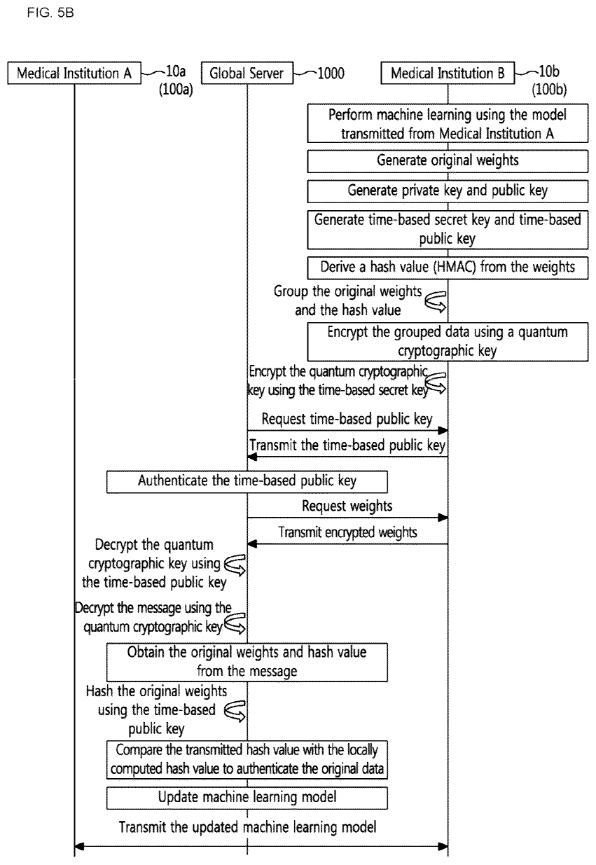

FIG. 5 is a flowchart that specifically illustrates the federated learning process described in FIG. 4. That is, it exemplifies a case in which two medical institutions (100a, 100b) participate in federated learning. Among the two medical institutions, Medical Institution A (100a) is configured as the institution that develops the machine learning model and initiates the federated learning process, while the other Medical Institution B (100b) is configured as the institution that receives the machine learning model developed by Medical Institution A (100a), performs machine learning, and generates weights. Medical Institution B (100b) may include at least one or more such institutions. In addition, the medical institutions (100a, 100b) may each represent a local server that is either internally equipped within, or connected to, the respective institution. Accordingly, in the embodiment described below, references to 100a and 100b may be understood as denoting Medical Institutions A and B, respectively, or as referring to the local servers thereof.

FIG. 5A is a flowchart illustrating the federated learning process between Medical Institution A (100a) and the global server (1000).

Referring to FIG. 5A, Medical Institution A (100a) collects medical data through a data acquisition unit (110) and performs data clustering learning based on the distribution characteristics of the collected medical data, in the case where the medical data follows a non-independent identically distributed (non-IID) pattern. The hierarchical clustering learning method for alleviating data heterogeneity has been described in detail in FIG. 2 and will thus be omitted here. If the medical data follows an independent identically distributed (IID) pattern, the hierarchical clustering learning process need not be performed.

Thereafter, the learning unit (142) performs machine learning using the collected medical data to predict patient prognosis and generates original weights as a result of the machine learning. At this time, the cryptographic key generation unit (147) generates a private key and a public key, and also generates a time-based secret key and a time-based public key based on the time of occurrence of the weights. As described above, the time of occurrence of the weights is converted into a timestamp code, which is generated in a form that can be combined with the private key and the public key; in this state, the private key is combined with the timestamp code to generate a time-based secret key, and the public key is combined with the timestamp code to generate a time-based public key.

Then, the time-based public key is combined with the original weights to generate a hash value. The generated hash value and the original weights are grouped together and encrypted using a quantum cryptographic key. Subsequently, the quantum cryptographic key is encrypted again using the time-based secret key.

Medical Institution A (100a), in response to a request for a time-based public key from the global server (1000), transmits the time-based public key at a predetermined communication time based on the timestamp code. The global server (1000) authenticates the time-based public key by comparing the communication time code of the time-based public key with the actual time of communication.

If the time-based public key is successfully authenticated, the global server (1000) requests the weights from Medical Institution A (100a) and receives the encrypted weights. The global server (1000) then decrypts the encrypted quantum cryptographic key using the time-based public key. Subsequently, using the quantum cryptographic key that was previously distributed, the global server decrypts the original weights and the hash value, which were encrypted with the quantum cryptographic key, thereby obtaining the original weights and the hash value.

The global server (1000) hashes the original weights using the time-based public key and performs source authentication by comparing the result with the hash value received from Medical Institution A (100a). If the source authentication is successfully performed, the global server (1000) transmits the machine learning model sent by Medical Institution A (100a) to Medical Institution B (100b).

FIG. 5B is a flowchart illustrating the federated learning process between Medical Institution B (100b) and the global server (1000).

In accordance with the process of FIG. 5A, when Medical Institution B (100b) receives the machine learning model transmitted by Medical Institution A (100a), Medical Institution B (100b) uses the machine learning model to perform learning on the medical data it has collected and generates original weights.

After generating the original weights, the process proceeds in the same manner as described in FIG. 5A. That is, a private key and a public key are generated, and a time-based secret key and a time-based public key are generated based on the time of occurrence of the weights. Then, the time-based public key is combined with the original weights to generate a hash value. The generated hash value and the original weights are grouped together, encrypted using a quantum cryptographic key, and the quantum cryptographic key is subsequently encrypted again using the time-based secret key. Thereafter, in response to a request for the time-based public key from the global server (1000), Medical Institution B (100b) transmits the time-based public key at a predetermined communication time based on the timestamp code. The global server (1000) authenticates the time-based public key by comparing the communication time code of the time-based public key with the actual time of communication.

If the time-based public key is successfully authenticated, the global server (1000) requests the weights from Medical Institution B (100b) and receives the encrypted weights. The global server then decrypts the encrypted quantum cryptographic key using the time-based public key, and subsequently decrypts the original weights and the hash value-encrypted with the quantum cryptographic key-using the previously distributed quantum cryptographic key, thereby obtaining the original weights and the hash value.

The global server (1000) hashes the original weights using the time-based public key and performs source authentication by comparing the result with the hash value received from Medical Institution B (100b). If the source authentication is successfully performed, the global server (1000) updates the machine learning model and transmits the updated machine learning model to both Medical Institution A (100a) and Medical Institution B (100b).

This process is continuously repeated, and as the number of iterations increases, the machine learning model is progressively updated and optimized.

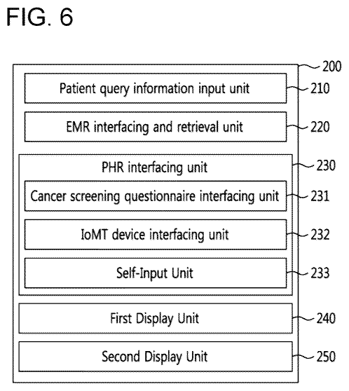

FIG. 6 is a block diagram illustrating the configuration of a terminal device that operates in conjunction with the local servers (100a to 100n) according to an embodiment of the present invention. The terminal device (200) may be a personal computer (PC) or a portable device that can be carried by medical personnel. The terminal device (200) may provide a visualized medical information service that can be utilized by medical personnel in actual clinical settings. As an example of the medical information service, the terminal may display analysis results of the federated learning process and, in addition to patient data within the medical institution, may interlink various healthcare data to provide patient-specific health management information.

Referring to FIG. 6, the terminal device (200) includes a patient query information input unit (210), an EMR interfacing and retrieval unit (220), a PHR interfacing unit (230), a first display unit (240), and a second display unit (250).

The patient query information input unit (210) is a unit through which medical personnel input a patient's personal information in order to retrieve the patient's medical history during a medical examination.

The EMR interfacing and retrieval unit (220) is a unit that, based on the patient query information entered, interfaces with the EMR backup server within the medical institution to retrieve the patient's historical health information related to previous hospital visits. From the linked EMR data, specific numerical data items used for prognosis prediction analysis (e.g., creatinine levels, hemoglobin levels, etc.) may be provided through the user interface of the health information application service.

The PHR interfacing unit (230) includes: a cancer screening questionnaire interfacing unit (231), which provides a method for either allowing medical personnel to directly input documented cancer/health screening survey results, or for automatically interfacing, in real time, cancer/health screening surveys that have been self-entered by the patient using a separate mobile device; an IoMT device interfacing unit (232), which acquires and interfaces the most up-to-date health status information of the patient (e.g., body composition, physical activity, blood pressure, blood glucose, heart rate, body temperature, etc.) measurable via wearable devices or medical Internet of Things (IoMT) devices used at home; and a self-input unit (233), which allows medical personnel using the health information application service to manually input additional health information deemed necessary during patient examinations. The data entered through the self-input unit (233) is typically in a free-text format that varies by user, making it difficult to utilize as a variable (feature) in a prognosis prediction analysis model that requires a standardized data format. However, it may serve as a useful reference for generating personalized medical content (e.g., home-based health management recommendations, etc.).

The first display unit (240) is a unit configured to analyze, process, and output disease risk information predicted based on personalized health checkup data, and the second display unit (250) is a unit configured to provide personalized medical content information based on patient-specific health information. For example, the second display unit (250) may provide, through a health information application service interface, an indication of whether the patient's blood pressure falls within a normal range by comparing it with the blood pressure data of other users. In another example, based on analysis results from a federated learning-based disease prognosis prediction model, and in response to a determination that the patient is at elevated health risk for a particular disease due to an increase or decrease in individual numerical indicators, the second display unit (250) may provide a medical content information service including at least one of a personalized dietary recommendation service, exercise recommendation service, supplement recommendation service, or a content linking service for mediating connection with external institutional server devices.

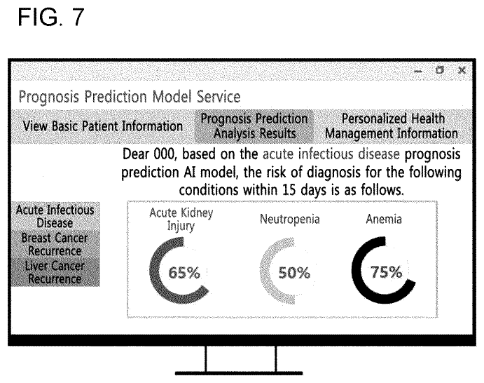

FIG. 7 is a diagram illustrating the configuration of a user interface screen displayed on the first display unit (240) according to the present invention, and represents the results of prognosis prediction analysis.

As shown therein, it can be seen that prognosis prediction results for each disease are provided based on the results of federated learning using the machine learning model described above. In the drawing, as an example of prognosis prediction analysis for an acute infectious disease, the probabilities of developing acute kidney injury, neutropenia, and anemia within 15 days are presented. When a different disease is selected, corresponding analysis results are provided accordingly.

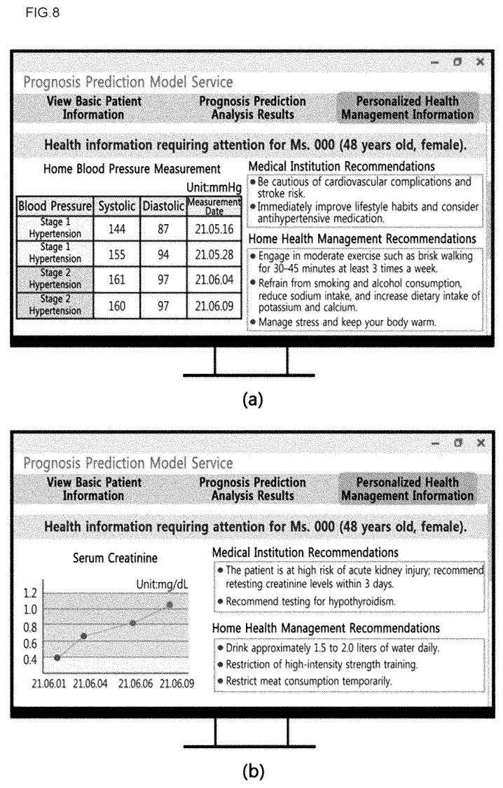

FIG. 8 is a diagram illustrating the configuration of a user interface screen displayed on the second display unit (250) according to the present invention, and represents personalized health management information. As shown therein, based on changes in serum creatinine levels and blood pressure status, the system provides both institution-based recommendations and home-based health management guidance in a manner that allows the patient to easily recognize and understand the information.

The patient may be able to manage their health in a personalized manner based on such recommendation information.

While the present invention has been described with reference to the illustrated embodiments, such embodiments are merely exemplary and not limiting. It will be apparent to those of ordinary skill in the art that various modifications, alterations, and equivalent embodiments can be made without departing from the spirit and scope of the present invention. Accordingly, the true technical scope of protection of the present invention should be defined by the spirit of the appended claims.

### INDUSTRIAL APPLICABILITY

The present invention may be implemented in medical institutions and other facilities in which various types of medical data are processed in an encrypted manner.

1. A system, comprising:
a plurality of local servers provided in medical institutions; and
a global server configured to communicate with the local servers,
wherein each of the local servers comprises a weight update unit configured to update its own machine learning model using weights from other local servers,
wherein the weight update unit is configured to apply weights differently based on whether medical data collected by each of the medical institutions follows an independent identically distributed distribution or a non-independent identically distributed distribution.

2. The system of claim 1,
wherein the weight update unit is configured to update weights using a hierarchical clustering method when the collected medical data exhibits characteristics of a non-independent identically distributed distribution,
wherein the hierarchical clustering method comprises:
calculating similarity between local servers using a first equation (1),
$$cos = \frac{{w}_{i}^{k - 1} \cdot {w}_{j}^{k - 1}}{‖ {w}_{i}^{k - 1} ‖ ‖ {w}_{j}^{k - 1} ‖} , \qquad ( 1 )$$
to cluster local weights having similar data distributions;
calculating similarity between different clusters using a second equation (2),
$$\frac{{n}_{A} {n}_{B}}{{n}_{A} + {n}_{B}} {‖ {w}_{A} - {w}_{B} ‖}^{2} , \qquad ( 2 )$$
to merge similar clusters; and
updating weights within each of the clusters using a third equation (3),
$${w}_{\text{?} + 1} ← {\sum }_{k = 1}^{K} \frac{{n}_{k}}{n} {w}_{\text{?} + 1}^{k} . \qquad ( 3 )$$
$$\text{?} \text{indicates text missing or illegible when filed}$$

3. The system of claim 1,
wherein the weight update unit is configured to update the weights of the machine learning model trained at each of the local servers using equation (3)
$${w}_{t + 1} ← {\sum }_{k = 1}^{K} \frac{{n}_{k}}{n} {w}_{t + 1}^{k} , \qquad ( 3 )$$
when the collected medical data exhibits characteristics of an independent identically distributed distribution.

4. The system of claim 1,
wherein the non-independent identically distributed distribution includes cases in which:
same data variables do not follow a uniform distribution across the medical institutions;
a difference in distribution between normal group and disease group for a target condition is observed across the medical institutions, such that the data does not follow a uniform distribution;
distribution of age (x) given disease (y) or distribution of disease (y) given age (x), based on conditional probability, does not follow a uniform distribution across medical institutions; or
an amount of data collected across medical institutions exhibits non-uniform distribution characteristics.

5. The system of claim 1,
wherein the local server further comprises a cryptographic key generation unit that utilizes a quantum cryptographic key and a timestamp code,
wherein the cryptographic key generation unit is configured to generate a time-based secret key and a time-based public key by respectively combining the timestamp code with a private key and a public key, and
wherein the time-based public key is transmitted to the global server.

6. The system of claim 5,
wherein a quantum key generation and distribution device is connected to at least one of the local server and the global server via a quantum key management device, and
wherein the quantum key generation and distribution device is configured to provide the quantum cryptographic key to the quantum key management device.

7. The system of claim 5,
wherein the local server further comprises a personal information protection unit configured to group weights generated based on a machine learning result and a hash value, encrypt grouped data using the quantum cryptographic key, and encrypt the quantum cryptographic key using the time-based secret key.

8. The system of claim 5,
wherein the timestamp code includes:
a weight occurrence time; and
communication time information for performing communication with the global server.

9. The system of claim 5,
wherein the global server is configured to: compare the timestamp code with an actual reception time of the time-based public key to authenticate the time-based public key; decrypt the quantum cryptographic key using the authenticated time-based public key; and decrypt the weights and a hash value using the decrypted quantum cryptographic key to obtain the weights and the hash value.

10. The system of claim 1,
wherein the local server further comprises:
a data acquisition unit configured to acquire medical data;
a common data model construction unit configured to transform heterogeneous data structures specific to each medical institution into a standardized model;
a data preprocessing unit configured to preprocess data required for machine learning from among data constructed based on the common data model; and
a learning unit configured to perform machine learning on the preprocessed data using the machine learning model.

11. A disease prognosis prediction system system, comprising:
the system of claim 1; and
a terminal device configured to interact with the local server,
wherein the disease prognosis prediction system is configured to analyze and predict a prognosis of a patient's disease based on the machine learning result.

12. The disease prognosis prediction system of claim 11,
wherein the terminal device comprises:
a patient query information input unit;
an EMR interfacing and retrieval unit configured to interwork with an EMR backup server within a medical institution to retrieve patient's historical health information;
a PHR interfacing unit including:
a cancer screening questionnaire interfacing unit,
an Internet of Medical Things (IoMT) device interfacing unit configured to acquire health status information using a IoMT device, and
a self-input unit for manually inputting health information;
a first display unit configured to analyze, process, and output disease risk level information predicted based on personalized health screening data; and
a second display unit configured to provide personalized medical content information based on the patient's customized health information.

13. A method for transmitting and receiving medical data in a system comprising local servers and a global server, the method comprising:
distributing, by a quantum key generation and distribution device, a quantum cryptographic key to each of the local servers and the global server via a quantum key management device;
generating, by the local server, a time-based secret key and a time-based public key by respectively combining a timestamp code with a private key and a public key;
performing, by the local server, machine learning on the medical data using a machine learning model, and generating weights;
grouping, by the local server, original weights of the generated weights and a hash value, and encrypting grouped data using the quantum cryptographic key;
encrypting, by the local server, the quantum cryptographic key using the time-based secret key; and
transmitting, by the local server, the encrypted original weights and the hash value to the global server.

14. The method of claim 13, comprising, by the global server:
authenticating a time-based public key transmitted by the local server;
decrypting, when the time-based public key is successfully authenticated, the quantum cryptographic key using the time-based public key;
decrypting the original weights and the hash value using the decrypted quantum cryptographic key to obtain the original weights and the hash value;
calculating a hash value of the original weights using the time-based public key, performing a comparison operation between the calculated hash value and a hash value received from a medical institution to authenticate the original weights, and updating the weights after the authentication; and
transmitting the weights to the local server to allow the local server to update the machine learning model.

15. The method of claim 14,
wherein authenticating the time-based public key by the global server is performed by comparing the timestamp code with an actual reception time of the time-based public key.

16. The method of claim 13, further comprising:
applying, by the local server, different weights based on whether the medical data collected by each medical institution follows an independent identically distributed distribution or a non-independent identically distributed distribution.

17. The method of claim 16,
wherein the local server updates the weights using a hierarchical clustering method when the collected medical data exhibits characteristics of a non-independent identically distributed distribution,
wherein the hierarchical clustering method comprises: calculating similarity between local servers using a first equation (1),
$$cos = \frac{{w}_{i}^{k - 1} \cdot {w}_{j}^{k - 1}}{‖ {w}_{i}^{k - 1} ‖ ‖ {w}_{j}^{k - 1} ‖} , \qquad ( 1 )$$
to cluster local weights with similar data distributions; calculating similarity between different clusters using a second equation (2),
$$\frac{{n}_{A} {n}_{B}}{{n}_{A} + {n}_{B}} {‖ {w}_{A} - {w}_{B} ‖}^{2} , \qquad ( 2 )$$
to merge similar clusters; and updating weights within each cluster using a third equation (3),
$${w}_{t + 1} ← {\sum }_{k = 1}^{K} \frac{{n}_{k}}{n} {w}_{t + 1}^{k} . \qquad ( 3 )$$

18. The method of claim 16,
wherein, when the collected medical data exhibits characteristics of an independent identically distributed distribution, the local server updates the weights of the machine learning models trained by each local server using equation (3)
$${w}_{t + 1} ← {\sum }_{k = 1}^{K} \frac{{n}_{k}}{n} {w}_{t + 1}^{k} . \qquad ( 3 )$$
